(19) United States
(12) Patent Application Publication
(10) Pub. No.: US 2005/0215826 A1
(43) Pub. Date: Sep. 29, 2005

(54) Process for preparing dimethylbenzophenones

(75) Inventors: Anand P. Singh, Pune (IN); Shainaz M. Landge, Pune (IN)

Correspondence Address: DARBY & DARBY P.C., P. O. BOX 5257, NEW YORK, NY, 10150-5257, US

(73) Assignee: Council of Scientific and Industrial Research, New Delhi (IN)

(21) Appl. No.: 10/811,237

(22) Filed: Mar. 26, 2004

(51) Int. Cl. C07C 45/46

(52) U.S. Cl. 568/319

(57) ABSTRACT

The present invention provides a process for preparing 4,4′-dimethylbenzophenone of formula (1) by reacting toluene with para-toluoyl chloride (p-T-CI) acylating agent over a solid acid triflic acid functionalized mesoporous zirconia catalyst.

### FIELD OF THE INVENTION

The present invention relates to process for preparing dimethylbenzophenones. More particularly, the present invention provides a process for preparing 4,4′-dimethylbenzophenone of formula (1) by reacting toluene with para-toluoyl chloride (p-T-CI) acylating agent over a solid acid triflic acid functionalized mesoporous zirconia catalyst.

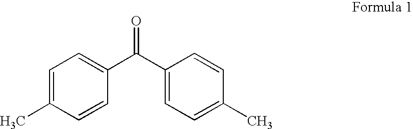

### BACKGROUND OF THE INVENTION

4,4′-dimethylbenzophenone is used mainly as a photosensitiser and applied to UV curable coating and inks. Disubstituted diphenylketones are also used as intermediates for pharmaceutical and agricultural chemicals. [U. Beck, in Ullmann's Encyclopedia of Industrial Chemistry, Eds. W. Gerhartz, Y. Y. Stephen, F. T. Campbell, R. Pfeffekorn and J. F. Rounsavifle, V C H, Weinheim, 1986, vol. A15, p.91]. 4,4′-dimethylbenzophenone gave high whiteness backgound and high d. images showing good resistance to plasticizers. [M Nakatsuka, Y. Tanabe, and K. Yoshikawa, JP 08290668 (1996)]. Also, 4,4′-dimethylbenzophenone is extensively used as UV light stablizers in plastics, cosmetics and films [M. Windholz (Ed.), Merck Index, An Encyclopedia of Chemical Drugs and Biochemicals, 1983, 10^{th} ed., published by Merck and co. Inc., Rahway, N.J., p. 7199]. The primary function is to protect the long-term degradation from all forms of wavelength of light and also helps in absorbing the UV radiation and prevents the formation of free radicals. [K. Beyer, W. Bergfeld, W. O. Berndt, R. Boutwell, W. Carlton, D. Hoffmann and A. L. schroeter, final report of the cosmetic ingredient review (1982-83), p. 25].

In the prior art, introduction of an acyl group in the aromatic nucleus (Toluene) using an acylating agent such as phosgene, or itself in the presence of a homogenous catalyst such as AICL₃ has resulted in lower yield of 4,4′-Dimethylbenzophenone [H. Limpricht, Annalen, 91,312, 1900; A. L. Klebanski, and K. K. Tchevychatova, Zh. Obshch. Khim; 5,535 (1935), C. A. 29,6879 (1935); R. E. Wilson and E. W. Fuller, J. Ind. Eng. Chem, 14,406 (1922)].

4,4′-dimethylbenzophenone has been synthesized by the reaction with toluene in presence of oxalyl chloride and Al₃ Cl₃ as a catalyst with yield of only 55% along with 33% of p-toluic acid as a side product. [H. A. Fahim, J. Chem. Soc. 520, (1949)]. 4,4′-dimethylbenzophenone has been synthesized by the reaction with toluene in presence of oxalyl chloride and Al₃Cl₃ as a catalyst with yield of only 55% along with 33% of p-toluic acid as a side product. [H. A. Fahim, J. Chem. Soc. 520, (1949)]. Other process includes the preparation of 4,4′-dimethylbenzophenones (4,4′-DMBP) by transcarbonylation reaction in which acylation of toluene with p-toluic acid in presence of polyp hosphoric acid (PPA) as a catalyst gives 4% yield. [R. C. Fusan, G. R. Barker, and B. Vittinnberga, J. Am. Chem. Soc, 81, 4858 (1959)].

Oxidation of Di-p-toluoyl methane with chromic acid in presence of acetic acid leads to formation of Di-p-toluoyl ketone along with p-toluic acid as side product, with comparable low yield of 4,4′-Dimethylbenzophenone. [H. Stefan, and W. F. Short J. Chem. Soc, 117, 510 (1920)]. Oxidation of p,p′-dimetyldiphenylmethane in presence of copper naphthenate yield 4,4′-dimethylbenzophenone [S. Kahn and N. J. Rutherford, U.S. Pat. No. 3,642,906 (1972)]. Recently, 4,4′-dimethylbenzophenone has been prepared by reacting p-toluic acid, toluene and various metals (Praseodymium, Dysprosium, Bismuth, Cerium, Thorium, Scandium and yttrium) triflate catalysts; 4,4′-dimethylbenzophenone being obtained in a yield in the range of 4-30% [M. Walker, U.S. Pat. No. 6,362,375 (2002)].

However, the above methods are disadvantageous from the industrial point of view, because the catalysts used are homogeneous. Thus large amount of base is required to neutralize the homogeneous catalysts.

Chemical industries are facing increasing pressure to reduce environmental impact. This is particularly true in the production of dimethyl phenylketones. Such reactions often require larg quantities of minerals or Lewis acid catalysts which are destroyed or diluted during the aqueous work-up procedures, leading to problems with equipment corrosion and expensive treatment. Furthermore, the reactions frequently use excess of reagents and expensive to treat. Furthermore, the reactions frequently use excess of reagents and are notoriously unselective. The overall result is excessive energy consumption, wastage of large quantities of homogeneous catalysts and excessive impact on the environment. An additional major handicap of the homogeneous catalysts is the difficulty of their disposal in an environmentally acceptable manner, after use in the benzoylation reaction of toluene.

Prior art processes for preparing dimethylbenzophenones suffer from several drawbacks, such as:
1. Use of AlCl₃ or homogeneious catalyst may result in side reactions of intra- or intermolecular migration of alkyl groups, dealkylation and transalkylation reactions.
2. The processes usually result in corrosion of the reactor
3. Difficulty in removal of the catalyst AlCl₃ from the products
4. Use of stoichiometric amount of catalyst in all the methods described above. Some of them are hazardous and difficult to handle. In some cases catalyst is consumed during the reaction and in some cases catalyst are less selective.

### OBJECTS OF THE INVENTION

The main object of the present invention to provide an improved process for the production of 4,4′-dimethylbenzophenone by the benzoylation of toluene with para-toluoyl chloride in presence of triflic acid functionalized mesoporous zirconia catalysts.

Another object of the invention is to obviate the drawbacks and limitations of the prior art such as removal of HCl from the product during the reaction, avoiding side reactions occurring due to use of homogeneous catalysts.

Still another object of the invention is to provide an improved process for the preparation of dimethylbenzophenones, which uses non-hazardous solid acid catalyst.

A further object of the invention is to provide a process which leads to high yields and selectivity to 4,4′-dimethylbenzophenone resulting from good conversion of toluene.

### SUMMARY OF THE INVENTION

The objects of the present invention are achieved by using triflic acid functionalized mesoporous zirconia, since this catalyst proves to be good for benzoylation reactions. In accordance with the process of the present invention toluene can be converted to 4,4′-dimethylbenzophenone in the presence of triflic acid functionalized mesoporous zirconia catalyst using solution of an acylating agent and toluene with stirring in an oil bath.

Accordingly, the present invention provides a process for preparing 4,4′-dimethylbenzophenone of formula 1 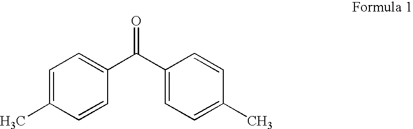
which comprises acylating toluene with an acylating agent in presence of a solid acid triflic acid functionalized mesoporous zirconia catalyst, and separating the product.

In one embodiment of the invention, the reaction is carried out for a time period in the range of 1 to 24 hours.

In another embodiment of the invention, the reaction is carried out at a temperature in the range of 100-150° C.

In one embodiment of the invention, the acylating agent is selected from halides of benzoic acids.

In another embodiment of this invention the trific acid functionalized mesoporous zirconia catalyst has the molar composition:
Zr(OC₄H₉)₄:BuOH:CTMABr:TMAOH:H₂O
Mesoporous Zr(OH)₄:Dry toluene:CF₃SO₃H
wherein Zr(OC₄H₉)₄ is Zirconium tetra butoxide, BuOH is 1-butanol, CTMABr is Cetyltrimethylammonium bromide, TMAOH is Tetramethylammonium hydroxide, Zr(OH)₄ is Zirconium tetra hydroxide, and CF₃ SO₃ H is triflic acid, having Zr (OH)₄/CF₃ SO₃ H molar ratio of from 5-30 and a pore size of 0.45-0.33 Å, and surface of 371-284 m2/g.

In another embodiment of the invention, the molar ratio of toluene to the acylating agent is in the range of 1:1 to 10:1.

### DETAILED DESCRIPTION OF THE INVENTION

In view of the above mentioned drawbacks of homogeneous catalysts in the prior art process, it was found desirable during the course of the research work leading the present invention to develop an environmentally acceptable solid selective, regeneratable and recyclable catalyst for the production of diphenyl ketone and particularly 4,4′-dimethylbenzophenone in high selectivity

In the process of the present invention, it is essential to use the solid acid catalyst having molar ratio of from 5-30 and a pore size of 0.4-0.33 Å. When a solid acid catalyst with molar ratio or a pore size being outside the above-mentioned range is used, the selectivity for the above mentioned products would be substantially poor.

A typical representative of solid acid catalyst, which satisfies the above-mentioned conditions, is triflic acid functionalized mesoporous zirconia catalyst, which has Zr (OH)₄/CF₃ SO₃ H molar ratio of from 5-30 and a pore size of 0.45-0.33 Å, and surface area of 371-284 m²/g:

The present invention provides an improved process for the production of 4,4′-dimethylbenzophenone formula-(1). 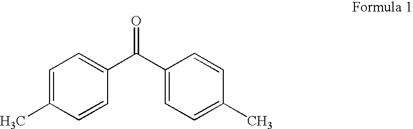
by the acylation of toluene with an acylating agent such as halides of benzoic acids (for example para-toluoyl chloride) and in the presence of a solid acid triflic acid functionalized mesoporous zirconia catalyst, at a temperature in the range of 100-150° c. for 1-24 h and separating the product by conventional methods to obtain the product.

The trific acid functionalized mesoporous zirconia catalyst used in the reaction has the molar composition as follows
Zr (OC₄H₉)₄:Bu OH:CTMABr:TMAOH:H₂O
Mesoporous Zr (OH)₄:Dry toluene:CF₃ SO₃ H

(where Zr (OC₄H₉)₄ is Zirconium tetra butoxide, BuOH is 1-butanol, CTMABr is Cetyltrimethylammonium bromide, TMAOH is Tetramethylammonium hydroxide, Zr (OH)₄ is Zirconium tetra hydroxide, and CF₃ SO₃ H is triflic acid, having Zr (OH)₄/CF₃ SO₃ H molar ratio of from 5-30 and a pore size of 0.45-0.33 Å, and surface of 371-284 m2/g and characterized by the X-ray diffraction pattern and infrared spectral data which are presented in Table—1,2,3 abd 4 respectively.

TABLE 1
X-ray diffraction data of mesoporous Zr (OH)₄

| 2 Time | Relative |
|---|---|
| Theta | Intensive I/I₀ |
| 4.7 + .1 − 0.2 | 90.1 |
| 32.2 + 0.2 − 0.1 | 100.00 |
| 50.1 + 0.2 − 0.2 | 94.2 |
| | 94.2 |

TABLE 2
X-ray diffraction data of triflic acid functionalised
mesoporous ziconia catalyst.

| 2 Time | Relative |
|---|---|
| Theta | Intensive I/I₀ |
| 4.7 + .1 − 0.2 | 71.2 |
| 32.2 + 0.2 − 0.1 | 100.0 |
| 50.1 + 0.2 − 0.2 | 97. |

TABLE 3
Infrared spectroscopic data of mesoporous Zr (OH)₄

| Frequency (cm⁻¹) | Relative Intensive |
|---|---|
| 3549 | vs & b |
| 2380 | s |
| 1610 | vs |
| 1585 | w |
| 940 | w |

TABLE 4
Infrared spectroscopic data of triflic acid functionalised
s zirconia catalyst

| Frequency (cm⁻¹) | Relative Intensive |
|---|---|
| 3549 | vs & b |
| 1620 | vs |
| 1296 | vs |
| 1184 | sh |
| 1043 | s |
| 800 | s & b |
| 601 | s |

The molar ratio of toluene to acylating agents may be from 1:1 to 10:1

In a feature of the present invention there is no risk of explosion. Thus, the process of the present invention is to effectively acylate the para-position of the substate while suppressing the acylation at the ortho and Meta position. The present invention is describe in a further detail with reference to the following examples, which should not be, however constructed to limit the present invention in any manner whatsoever.

### EXAMPLE—I

This example illustrates the procedure for the preparation of a typical mesoporous Zr (OH)4. It was carried out using the following gel composition and procedure. 0.07 Zr (OC4H9)4:1.4 Bu OH: 0.02 CTMA Br: 0.014 TMAOH: 1.7 H2O

A mixture of Zirconium (IV) butoxide and 1-butanol was stirred. Then the required amount of water was added dropwise into this mixture under stirring. Further, the precipitated Zr (OH)4 mixture was added to aqueous solution of CTMABr, and TMAOH, under continuous stirring. After further stirring for 2 h. the surfactant from the synthesized material was removed by the extraction with a mixture containing ethanol and HCI per gram of the solid material under reflux condition for 48 h. Thus mesoporous Zr (OH)₄ was washed and dried at 373 K for 2 h. the X-ray diffraction and framework IR data of the so obtained material corresponds to those given in Table 1 and Table 3, respectively.

### EXAMPLE—2

This example illustrates the procedure for the synthesis of mesoporous ═Zr—O—SO₂-CF₃. The resulting solid mesoporous material, Zr (OH)₄, (example—1) was functionalized with triflic acid by post-synthesis procedure using the molar composition. 0.07 Mesoporous Zr (OH)₄: 0.7 dry toluene: 0.03 CF₃SO₃H

Triflic acid (0.03 mol, Lancaster, UK) was added drop wise into the mixture of toluene and mesoporous Zr(OH)4 at 363 K under nitrogen atmosphere; then it was further refluxed for 2 h. Next the sample was cooled, filtered, washed with acetone and dried at 373 K for 6 h. The Soxhlet extraction of the material was carried out at 348 K for 24 h using a mixture of dichloromethane and diethyl ether 100 g each per gram of the catalyst. Then the sample was dried at 473 K for 10 h. the X-ray diffraction and framework IR data of the so obtained material corresponds to those given in Table 2 and Table 4, respectively.

### EXAMPLE—2

This example illustrates the procedure for the benzoylation of toluene to 4,4′-dimethylbenzophenone. 1.075 g (0.01 mol) of toluene, 1.54 g (0.01 mol) of para-toluoyl chloride and 10 ml of nitro benzene (as taken) in a two-necked round bottom flask. 0.5 g of triflic acid functionalized mesoporous zirconia catalyst was added in the reaction mixture. The reaction mixture was heated up to 403 K with stirring. The reaction was continued for 24 h. the reaction mixture was cooled down to room temperature and analyzed with gas chromatograph. The results are recorded in Table 5.

TABLE 5
Benzoylation of toluene with para-toluoyl chloride over triflic acid
functionalized mesoporous zirconia catalyst after 24 h.

| | Reaction time (h) |
|---|---|
| | 24 |
| Conversion of Para-toluoyl chloride (wt. %) | 82.1 |
| Product distribution (wt. %) | |
| 4,4′-dimethylbenzophenone (4,4′-DMBP) | 74.1 |
| 2,4′-dimethylbenzophenone (2,4′-DMBP) | 21.4 |
| Other | 4.5 |
| Turn over frequency (TOF) | |
| (10-1 h-1 mol-1 S) | 7.8 |
| 4,4′-DMBP/2,4′-DMBP | 3.4 |

### EXAMPLE—4

This example illustrates the effect of reaction time on the benzoylation of toluene to 4,4′-dimethylbenzophenone over 24 hr period. 1.075 g (0.01 mol) of toluene, 1.54 g (0.01 mol) of para-toluoyl chloride and 10 ml of nitro benzene (as solvent) were taken in a two-necked round bottom flask. 0.5 g of triflic acid fictionalized mesoporous zirconia catalyst was added in the reaction mixture. The reaction mixture was heated up to 403 K with stirring. The reaction was continued for 24 h. the reaction mixture was cooled down to room temperature and analyzed with gas chromatograph. The results are recorded in Table 6.

TABLE 6
Influence of reaction time on benzoylation of toluene with para-toluoyl
chloride over triflic acid functionalized mesoporous zirconia catalyst
over 24 h period.

| | Reaction time (h) | | | | | |
|---|---|---|---|---|---|---|
| | 1 | 2 | 4 | 8 | 22 | 24 |
| Conversion of Para-toluoyl | 19.7 | 28.6 | 45.1 | 80.7 | 76.0 | 82.1 |
| chloride (wt. %) | | | | | | |
| Product distribution (wt. %) | | | | | | |
| 4,4′-dimethylbenzophenone | 78.4 | 78.2 | 76.1 | 73.5 | 74.2 | 74.1 |
| (4,4′-DMBP) | | | | | | |
| 2,4′-dimethylbenzophenone | 21.6 | 21.8 | 20.0 | 22.5 | 21.5 | 21.4 |
| (2,4′-DMBP) | | | | | | |
| Other | — | — | 3.9 | 4.0 | 4.3 | 4.5 |
| Turn over frequency (TOF) | 45.2 | 32.8 | 25.8 | 14.5 | 7.9 | 7.8 |
| (10-1 h-1 mol-1 S) | | | | | | |
| 4,4′-DMBP/2,4′-DMBP | 3.6 | 3.5 | 3.8 | 3.2 | 3.4 | 3.4 |

### EXAMPLE—5

This example illustrates the effect of various concentration of catalyst=Catalyst/p-t-Ci (wt./wt.) ratio on triflic acid functionalized mesoporous ziconia catalyst on the benzoylation of toluene to 4,4′-dimethyl benzophenone. The Catalyst/p-T-Ci (wt./wt.) ratio was changed by varying the amount of catalyst and keeping p-t-ck weight constant. 1.075 g (0.01 mol) of toluene, 1.54 g (0.01 mol) of para-toluoyl chloride and 10 ml of nitro benzene (as solvent) were taken in a two -necked round bottom flask. And various Catalyst/p-t-ci (wt./wt.) ratio (0,0.06, 0.19, 0.32, 0.45) were added in the reaction mixture was heated up to 403 K with stirring The result are recorded in table 7.

The reaction was continued for 24 h. The reaction mixture was cooled down to room temperature and analyzed with gas chromatograph. The result are recorded in table 7.

TABLE 7
Influence of the amount of Catalyst/p-T-PI (wt./wt.) ratio on
benzoylation of toluene with para-toluoyl chloride over
triflic acid functionalized mesoporous zirconia catalyst after 24 h.

| | Reaction time (h) | | | | |
|---|---|---|---|---|---|
| | No Cata | 0.06 | 0.19 | 0.32 | 0.45 |
| Conversion of Para-toluoyl | — | 9.3 | 48.3 | 82.1 | 87.9 |
| chloride (wt. %) | | | | | |
| Product distribution (wt. %) | | | | | |
| 4,4′-dimethylbenzophenone | — | 76.8 | 75.8 | 74.1 | 74.5 |
| (4,4′-DMBP) | | | | | |
| 2,4′-dimethylbenzophenone | — | 23.2 | 20.3 | 21.4 | 21.5 |
| (2,4′-DMBP) | | | | | |
| Other | — | — | 3.9 | 4.5 | 4.0 |
| Turn over frequency (TOF) | — | 4.4 | 7.7 | 7.8 | 6.0 |
| (10⁻¹h⁻¹mol⁻¹S) | | | | | |
| 4,4′-DMBP/2,4′-DMBP | — | 3.3 | 3.7 | 3.4 | 3.4 |

### EXAMPLE—6

This example illustrates the effect of different reaction temperatures on the benzoylation of toluene to 4,4′-dimethyl benzophenone. Over triflic acid functionalized mesoporous zirconia catalysts. 1.075 g (0.01 mol) of toluene, 1.54 g (0.01 mol) of para-toluoyl chloride and 10 ml of nitro benzene (as solvent) were taken in a two-necked round bottom flask. And 0.5 g of catalyst was added in the reaction mixture. The reaction was carried out at various temperatures (383, 393 403, 413 K) with stirring. The reaction was continued for 24 h. the reaction mixture was cooled down to room temperature and analyzed with gas chromatograph. The results are recorded in table 8.

TABLE 8
Influence of the reaction temperature on the benzoylation of
toluene with para - toluoyl chloride over triflic acid
functionalized mesoporous zirconia catalyst after 24 h

Temperature (K)

| | 383 | 393 | 403 | 413 |
|---|---|---|---|---|
| Conversion of Para-toluoyl | 55.4 | 67.7 | 82.1 | 88.2 |
| chloride (wt. %) | | | | |
| Product distribution (wt. %) | | | | |
| 4,4′-dimethylbenzophenone | 74.8 | 76.1 | 74.1 | 73.3 |
| (4,4′-DMBP) | | | | |
| 2.4′ dimethylbenzophenone | 21.3 | 20.2 | 21.4 | 22.1 |
| (2,4′-DMBP) | | | | |
| Other | 3.9 | 3.7 | 4.5 | 4.6 |
| Turn over frequency (TOF) | 5.3 | 6.4 | 7.8 | 8.4 |
| (10⁻¹h⁻¹mol⁻¹S) | | | | |
| 4,4′-DMBP/2,4′-DMBP | 3.5 | 3.7 | 3.4 | 3.3 |

The process of present invention show remarkable high industrial merits over prior art process for the preparation of 4,4′-dimethylbenzophenone in high selectivity. The starting materials are easily available and easy to handle and that 4,4′-dimethylbenzophenone can be produced in high yield by extremely simple operation.

Another important and advantageous feature of the process of the present invention is the use of the non-hazardous solid triflic acid functionalized mesoporous Zirconia catalysts.

Another important and advantageous feature of the process of the present invention is that it does not pose any risk of explosion. The most important and advantageous feature of the process of the invention is that both the yield and selectivity to 4,4′-dimethyl benzophenone are very high.

The use of catalyst in the liquid phase organic of the present invention provides the following advantages:
1. Easy separation of the product from the solid by means of a simple procedure of filtration.
2. Adsorption or inclusion of substrate and reagent molecule into the pores of solids with nanometer dimension organizes in the molecules in close proximity to lower the activation energy of the reaction.
3. Well-defined crystalline, structure, uniform meso cavities effecting selecting reactions of organic incorporated therein under restriction.

1. A process for preparing 4,4′-dimethylbenzophenone of formula 1 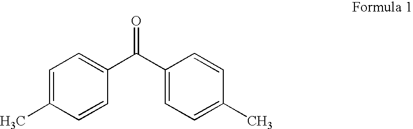
which comprises acylating toluene with an acylating agent in the presence of a solid acid triflic acid functionalized mesoporous zirconia catalyst, and separating the product obtained.

2. A process as claimed in claim 1 wherein the reaction is carried out for a time period in the range of 1 to 24 hours.

3. A process as claimed in claim 1 wherein the reaction is carried out at a temperature in the range of 100-150° C.

4. A process as claimed in claim 1 wherein the acylating agent is selected from halides of benzoic acids.

5. A process as claimed in claim 1 wherein the trific acid functionalized mesoporous zirconia catalyst has the molar composition:
Zr(OC₄H₉)₄:BuOH:CTMABr:TMAOH:H₂O
Mesoporous Zr(OH)₄:Dry toluene:CF₃SO₃H
wherein Zr(OC₄H₉)₄ is Zirconium tetra butoxide, BuOH is 1-butanol, CTMABr is Cetyltrimethylammonium bromide, TMAOH is Tetramethylammonium hydroxide, Zr(OH)₄ is Zirconium tetra hydroxide, and CF₃ SO₃ H is triflic acid, having Zr (OH)₄/CF₃ SO₃ H molar ratio of from 5-30 and a pore size of 0.45-0.33 Å, and surface of 371-284 m2/g.

6. A process as claimed in claim 1 wherein the molar ratio of toluene to the acylating agent is in the range of 1:1 to 10:1.

7. A process as claimed in claim 4 wherein the acylating agent comprises para-toluoyl chloride.
